(19) United States
(12) Patent Application Publication
(10) Pub. No.: US 2024/0342330 A1
(43) Pub. Date: Oct. 17, 2024

(54) METHOD FOR THE PREPARATION OF SURFACES, PARTICULARLY OF CARDIAC PROSTHESIS

(71) Applicant: BIOCOMPATIBILITY INNOVATION S.R.L., Este (Padova) (IT)

(72) Inventors: Filippo NASO, Este (Padova) (IT); Alessandro GANDAGLIA, Este (Padova) (IT); Ugo STEFANELLI, Este (Padova) (IT)

(21) Appl. No.: 18/698,719

(22) PCT Filed: Oct. 6, 2022
(86) PCT No.: PCT/IB2022/059550

§ 371 Date: Apr. 4, 2024

(30) Foreign Application Priority Data
Oct. 6, 2021 (EP) 21201230.6
Oct. 8, 2021 (IT) 102021000025796

(51) Int. Cl. A61L 2/18; A61F 2/24; A61L 101/34; A61L 101/36; A61L 101/44

(52) U.S. Cl. CPC A61L 2/18; A61F 2/24; A61F 2240/001; A61L 2101/34; A61L 2101/36; A61L 2101/44; A61L 2202/21

(57) ABSTRACT

A method for preventing both active and passive early degeneration of prosthesis to be contacted with biological fluid is provided.

### BACKGROUND OF THE INVENTION

The present invention finds application in the medical field and, in particular, in the preparation of biological surfaces to be in contact with bodily fluids.

Glutaraldehyde-fixed bioprosthetic heart valves (BHVs) have been reported to be prone to dystrophic calcification after mid-/long-term implantation in humans, with this being the main limiting factor affecting their longevity. Calcification is a complex and multi-factorial process not yet fully understood, which includes atherosclerosis-like tissue remodelling and prolonged exposure to mechanical stress. Among the causes responsible for calcific tissue dystrophy the treatment with glutaraldehyde (GLU) should also be taken into due consideration.

GLU is often used as the preferred fixative and sterilizing agent for many commercial bioprosthetic products, especially in surgical and transcatheter implantable heart valves (THVs). THV implant is considered a minimally invasive procedure that involves the use of a guide catheter for the positioning of the prosthesis, avoiding the opening-heart surgery. THVs may be an option for people who are at intermediate or high risk of complications from surgical heart valve replacement. Unfortunately, the GLU chemical instability is strictly involved in the exposure of potential calcium-binding sites (residual aldehydes, acids, Schiff bases, etc). As a result of interaction between tissue amino acid residues and GLU, negatively charged carboxylic acid groups can be created which can electrostatically interact with positively Ca²⁺ charged ions, becoming a tremendous attraction site for calcium. To make matters worse, even the free-to-react aldehyde groups can be easily oxidized into carboxylic residues via air, in-vivo blood and macrophage oxidation.

To decrease the influence on the calcification process, several changes in the GLU fixation protocols have been proposed by the BHVs manufacturers, including the addition of novel steps aimed at the chemical stabilization of the reactive aldehyde and carboxylic groups. GLU detoxification by urazole, diamine spacer extension, treatment by 2-amino oleic acid or incubation in ethanol are just some of the processes developed in the challenge of stabilizing GLU, with the hope of delaying the calcific tissue dystrophy.

Although the calcified degeneration of the BHVs is the long-term event generally responsible for the definitive failure of such biomedical devices, it must not be forgotten that there are a series of degenerative processes that begin to affect the prosthesis just a few hours after implantation, mainly compromising the structural aspect of the device.

Early structural BHV degeneration is a complex pathway due to multiple active and passive mechanisms closely related to each other. It is now well established as degenerative active mechanisms are triggered by early host's-response within the few hours after implantation often correlated to inflammation, subclinical leaflets thrombosis and/or bacterial infection. On the other side, passive deterioration is strictly related to graft fatigue resulting in holes formation, tearing and abrasion of the leaflets.

### Early Structural Degeneration—Passive Factors

As is well known, commercial BHVs do not constitute a viable tissue and, by definition, are not capable of extracellular matrix regeneration and remodelling so that any changes in the collagen meshwork (delamination, structural rearrangements, and destruction) resulting from cyclic loading are considered irreversible damages. Prolonged cyclic loading during accelerated wear testing, highlighted a marked decrease in radial extensibility because of the stiffening of the effective collagen fiber network. The stiffening of the leaflets over time, causing anomalies in the distribution of mechanical stress, leads to overloads especially in the bending areas and in the suture areas around the stent. Histologic evaluation of explanted BHVs has demonstrated that leaflet tears and disrupted collagen fibre bundles are characteristic of a high strain area, even in the absence of associated calcification.

### Early Structural Degeneration—Active Factors

Subclinical leaflet thrombosis occurred frequently in BHVs replacement, more commonly in THVs (frequency of 13%) than in surgical BHVs (4%). It involves 30% of the BHVs at 1 year after the implant. Such pathology shows clinical signs within the first 30 days after the implantation causing a reduction in the mobility of the leaflet. In patients with reduced leaflet motion (HALT—hypoattenuating leaflet thickening), the thickness of the leaflets was significantly altered with complete immobility of at least one valve cusp. The HALT development represents a mild form of valve dysfunction related to a thrombus, early calcification and/or degeneration of the valve leaflets.

Anticoagulation therapy (both Novel Oral Anti-coagulants NOACs and warfarin) is effective in reducing the HALT complications, but HALT recurred in 50% of the patients where anticoagulation was discontinued. It is important to note that dual antiplatelet therapy, the standard of care for transcatheter valve implant, resulted not effective in the prevention or treatment of subclinical leaflet thrombosis.

The bacterial infection is another worrying aspect that contributes to early BHVs degeneration, especially in the case of THVs. Infective endocarditis (IE) has a significant impact both on the population and patient management. In the USA there are 40,000 to 50,000 new cases/year, with average hospital charges over $120,000/patient. Notwithstanding the improvements in diagnosis and surgical intervention, the 1-year mortality from IE is unchanged in over 2 decades. In the cases that prolonged antibiotic treatment is not sufficient, surgical valve replacement is required.

The ability of specific bacteria to colonize BHVs is a crucial aspect for the future of heart valve replacements since THVs have shown good results also in mid and low-risk patients enormously expanding the number of people subjected to this type of minimally invasive intervention. The replacement of a degenerated surgical BHV is frequently performed adopting a valve-in-valve approach. In this case, a THV is deployed inside the dysfunctional surgical BHV without its preliminary removal. This could potentially lead to bacterial migration from the dysfunctional BHV to the new THV resulting in bacterial growth and tissue colonization.

Finally, regulatory agencies require that medical devices be tested for material-mediated pyrogenicity following “ISO 10993-11:2017 Biological evaluation of medical devices—Part 11: Tests for systemic toxicity”. The term pyrogen (Greek pyros: fire) defines fever-inducing substances. A pyrogenic response induced by a medical device may be due to several causes depending on the presence of so-called “material-mediated pyrogens”. One class of well-known and well-characterized exogenous pyrogens is the class of endotoxins. Endotoxins are lipopolysaccharide components present on the cell walls of Gram-negative bacteria. Another broad class of exogenous pyrogens are non-endotoxin pyrogens, which include substances such as lipoteichoic acid originating from Gram-positive bacteria, and other compounds originating from fungi, yeast, viruses, bacteria, and parasites. The third class of non-endotoxin pyrogens is that of material-mediated pyrogens. Although no formal definition of material-mediated pyrogens exists, it is thought that they may leach from medical device materials or surfaces. Material-mediated pyrogens may also stem from contamination introduced during manufacturing and packaging, such as residues from cutting fluids, mould releases, cleaning agents, and processing aids. Therefore, it is of fundamental importance to develop treatments capable of improving the outcomes of BHVs without introducing chemicals or contaminants responsible to rise pyrogenic reactions.

### SUMMARY OF THE INVENTION

The inventors of the present patent application have surprisingly found a method for preventing both the active and the passive early degenerative occurrences in a biological matrix (particularly of medical devices and even more particularly to cardiac prosthesis) achieving an unprecedented GLU stabilization, resistance both to surface platelet adhesion and fibrin release and microorganisms' surface colonization. Finally, such a method showed an interesting improvement effect on the biomechanical properties of the treated biological matrix.

### BRIEF DESCRIPTION OF THE FIGURES

FIG. 1—Percentage of free-to-react aldehyde and carboxylic groups decrease in polyphenolic-treated GLU fixed pericardial patches (n=16 for each type of chemical group determination).

FIG. 2—Percentage of thrombus accumulation in polyphenolic-treated and untreated (GLU) pericardial samples (n=6 for each type of chemical group determination).

FIG. 3—Ultimate Tensile Strength comparison between polyphenolic-treated and untreated samples (GLU, n=36 for each type of chemical group determination).

FIG. 4—Percentage of elongation comparison between polyphenolic-treated and untreated samples (GLU, n=36 for each type of chemical group determination).

FIG. 5—Young's Modulus correlation between F polyphenolic-treated and untreated samples (GLU, n=36 for each type of chemical group determination).

### OBJECT OF THE INVENTION

In a first object, the present invention discloses a method for the treatment of a surface to be contacted with biological fluids.

As per a preferred aspect, said surface is the surface of a medical device.

As per a more preferred aspect, said surface is the surface of a biological prosthesis, which can be a cardiac prosthesis.

In a second object, the present invention discloses a surface to be contacted with biological fluids obtained with the method of the invention.

As per a preferred aspect, said surface is the surface of a medical device.

As per a more preferred aspect, said surface is the surface of a biological prosthesis, which can be a cardiac prosthesis.

The medical device, the biological prosthesis and the cardiac prosthesis comprising the surface according to the invention do represent further objects of the invention.

In a third object, the present invention discloses a method for the treatment of a disease comprising the use of the medical device, the biological prosthesis or the cardiac prosthesis of the invention.

As per a preferred aspect, said disease is a heart disease.

As per an aspect, said disease is in a human, while per another aspect, said disease is in an animal.

A solution comprising a phenolic compound or a mixture of phenolic compounds to be used in the method of the present application represents a further object of the invention.

According to another object of the invention, it is disclosed a method for the preparation of the invention solution comprising a phenolic compound or a mixture of phenolic compounds.

In a still further object, the present invention discloses a kit for performing the invention method.

### DETAILED DESCRIPTION OF THE INVENTION

According to a first object, the present invention discloses a method for the treatment of a surface to be contacted with biological fluids.

A biological fluid within the purposes of the present invention is represented by blood, serum, plasma, vitreous gel, tears, urine, saliva, faeces; including synovial, peritoneal, pericardial, pleural and amniotic fluid.

A biological surface can be represented by a surface of human or animal origin.

In particular, surfaces of animal origin may have an equine, porcine or bovine origin and preferably have a porcine or bovine origin; such surfaces may be considered as biological matrices.

In particular, said surface is a surface of a medical device.

Medical devices according to the present invention may be represented by: cardiac valve, tendon, ligament, pericardium, muscular fasciae, dura mater, tympanic membrane, intestinal submucosa, cartilage, adipose and bone tissue, pelvic, abdominal, breast and dermal tissue.

As per another aspect of the invention, said surface is the surface of a biological prosthesis.

A biological prosthesis according to the invention may be represented by: a vessel, a cardiac valve, a tendon, a ligament, pericardium, muscular fasciae, dura mater, tympanic membrane, intestinal submucosa, cartilage, adipose and bone tissue, pelvic, abdominal, breast and dermal tissue.

In a preferred embodiment, the biological prosthesis according to the invention is represented by a cardiovascular prosthesis, such as a cardiac valve, or a pericardial tissue patch.

In a more preferred embodiment, a cardiac valve that can be treated according to the present invention is represented by a surgical heart valve.

In an even more preferred embodiment, the cardiac valve which can be treated according to the present invention is represented by a trans-catheter implantable heart valve; said valve requires to be implanted through a catheter and are folded to be housed within the catheter.

According to the method of the invention, the disclosed surface is contacted with a solution comprising a phenolic compound or a mixture of phenolic compounds.

For the purposes of the present invention, a phenolic compound shall be intended as a phenolic or polyphenolic compound (in some instances they both are referred as “phenolic” or “polyphenolic”, only) used here as synonyms) selected from the group comprising: phenols, phenolic aldehydes, phenolic acids, phenylamines, phenol compounds, flavonoids, phenylpropanoids and tannins.

In particular, a phenolic compound is selected in the group comprising: vanillin, cinnamic acids, phenylalanine, coumarins, xanthones, catechins, flavonons, flavones, chalcones, flavanonols, flavanols, leucoanthocyanidin, anthocyanidin, hydroxycinnamic acids.

More, in particular, a phenolic compound can be selected in the group comprising: resveratrol, aloin, cyanarin, epigallocatechin, tannic acid, caffeic acid, chlorogenic acid, hydroxytyrosol, rosmarinic acid, narigenin, gallic acid, hesperitin, quinic acid, eleonolic acid, pinoresinol, luteolin, apigenin, tangeritin, isorhamnetin, kaempferol, myricetin, eriodictyol, hesperetin, naringenin, theaflavin, thearubigins, daidzein, genistein, glycitein, pterostilbene, delphinidin, malvidin, pelargonidin, peonidin, chicoric acid, ferulic acid, salicylic acid.

For the purposes of the present invention, derivatives of the above-disclosed phenolic or polyphenolic compound are also encompassed; for instance, salts or esters or isomers may also be used.

In one embodiment of the invention, the solution of the invention comprises a mixture of two or more of the above-disclosed phenolic or polyphenolic compounds.

As per a preferred embodiment, the solution of the invention may comprise a mixture of two or more of the above-disclosed phenylpropanoids.

Here below there are reported some components as well as some solutions according to the invention:

| Solution | Component A | Component B |
|---|---|---|
| 1 | Resveratrol | Tannic Acid |
| 2 | Resveratrol | Cynarin |
| 3 | Aloin | Epigallocatechin |
| 4 | Aloin | Chlorogenic Acid |
| 5 | Caffeic Acid | Tannic Acid |
| 6 | Caffeic Acid | Hydroxytyrosol |
| 7 | Rosmarinic Acid | Cynarin |
| 8 | Naringenin | Gallic Acid |
| 9 | Hesperetin | Gallic Acid |

As per the preparation of the invention solution, a phenolic or polyphenolic compound is solubilized in an alcoholic solvent.

In case a mixture of the phenolic or polyphenolic compound is prepared, then the solution of each compound are prepared separately and then admixed together.

According to a preferred embodiment of the invention, the solution comprises a mixture of phenolic compounds and more preferably a mixture of phenylpropanoid compounds.

For said purposes, a first component is solubilized in an alcoholic solution (Component A), preferably 10% of the final volume of the solution.

An alcoholic solvent according to the invention may comprise methanol, ethanol, isopropanol, butanol, etc. and preferably comprises or it is represented by ethanol.

Within the solution of the invention, a second component is solubilized in an isotonic buffered solution (Component B), preferably 90% of the final volume of the solution.

In one embodiment of the invention, the final solution is a hydroalcoholic solution.

According to the present invention, in the disclosed method the solution of a phenolic compound or of a mixture of phenolic compounds preferably has a pH value of between 5 and 7.

Once it is prepared, the solution can optionally be filtered with a 0.22 μm filter.

As per the present invention, in the disclosed method the surface is contacted with the solution of a phenolic compound or of a mixture of phenolic compounds for a period of time of less than 2 hours.

Preferably, the contacting is continued for a period of about one hour.

More preferably, the contacting is continued for a period of about 30 minutes.

In an even more preferred embodiment, the contacting comprises a first step and a second step.

In a preferred embodiment, the first contacting step is performed for 30 minutes and the second contacting step is performed to 30 minutes.

Optionally, between the two contacting steps, a rinsing step (also referred to as a washing step) may be performed.

According to a preferred embodiment of the invention, the method is performed in the dark and more preferably completely in the dark (i.e. avoiding any exposure to light).

According to a preferred embodiment, the method is performed while stirring the solution.

As per the temperature of the contacting step, it is preferably performed at a temperature of about +20° C.±10° C.

In a preferred embodiment of the invention, after the contacting step, the treated surface, medical device, biological prosthesis or cardiac prosthesis may be subjected to one or more washing steps.

Preferably, each of said washing steps is performed with a suitable buffer; for example, a suitable buffer may be represented by a phosphate buffer.

In an embodiment, each washing step may be performed for a period of about 15-30 minutes.

In another embodiment, each washing step may be performed for a period of about 12-48 hours.

According to an embodiment of the present invention wherein the disclosed method is performed on a biological surface, said biological surface can be previously subjected to a pre-treatment step.

In particular, said pre-treatment step may have one or more of the following effects:
stabilizing proteins,
stabilizing or removing lipids,
stabilizing or removing cell structures,
lowering the antigenicity.

For the purposes of the present invention, said pre-treatment step may comprise a step of pre-treatment with one or more of glutaraldehyde, formaldehyde, quercetin or genipin as well as a treatment for the removal of phospholipids.

According to a particular embodiment of the invention, before subjecting the biological surface to the pre-treatment step, it can be subjected to a preparing step with a capping agent selected from the group comprising: glycerol, heparin, amines (i.e. alkyl amines, amino alcohols, ethanolamine), amino acids (lysine, hydroxylysine, amino sulfonates, taurine, amino sulfates, dextran sulfate, chondroitin sulfate), hydrophilic multifunctional polymers (i.e. polyvinyl alcohol, polyethyleneimine), hydrophobic multifunctional polymers (i.e. alpha-dicarbonyls, methylglyoxal, 3-deoxyglucosone, glyoxal), hydrazides (i.e. adipic hydrazide), N,N-disuccinimidyl carbonate, carbodiimide (i.e. 1-ethyl-3-3-dimethylamino propyl carbodiimide hydrochloride—EDC, N-cyclohexyl-N′-(2-morpholino ethyl)carbodiimide—CMC, 1,3-di-cyclohexyl carbodiimide—DCC, 2-chloro-1-methyl pyridinium iodide—CMPI, 2-chloro-1-methyl pyridinium iodide—CMPI), antibiotics, cell recruiting agents, hemocompatibility agents, anti-inflammatory agents, antiproliferative agents, reducing agents (i.e. sodium cyanoborohydride, sodium borohydride, sodium bisulfite+acetylacetone, formic acid—formaldehyde, mono-, di- or polyepoxy alkanes).

As per the present invention, the method for the treatment of a surface to be contacted with a biological fluid according to the invention is a stabilization method.

In particular, said stabilization method inactivates the available reacting groups on the pre-treated surface.

More in particular, said stabilization method inactivates the available aldehydic and carboxylic groups on the pre-treated surface.

As per the present invention, the disclosed method is an anti-calcific method.

A surface obtained with the method of the invention represents another object of the present application.

A biological prosthesis, a medical device and particularly a cardiac prosthesis comprising the surface obtained according to the method of the present application represent further objects of the present invention.

A surface obtained with the pre-treatment and the treatment method of the invention represents another object of the present application.

A biological prosthesis, a medical device and particularly a cardiac prosthesis comprising the surface obtained according to the pre-treatment and the treatment method of the present application represent further objects of the present invention.

As per the present invention, the method for the treatment of a surface to be contacted with a biological fluid according to the invention is a protective method.

In particular, said protective method prevents subclinical thrombosis occurrences.

More in particular, the method of the invention is an anti-platelet adhesiveness method.

Even more in particular, the method of the invention prevents the synthesis of fibrin.

The synthesis of fibrin is releated to the activation of soluble fibrinogen to insoluble fibrin polymers. Such polymers aggregate laterally to make fibers, which then branch to yield a three-dimensional network and, interacting with circulating platelet, lead to the formation of the fibrin clot essential for haemostasis and wound coagulation. If the clot enters the bloodstream it is called thrombus and it can obstruct small/medium-sized vessels causing ischemia, stroke and heart attack.

In particular, the protective method of the invention has proved to prevent and avoid the anchoring of circulating platelets on the biological surfaces treated and/or pre-treated according to the invention.

A surface obtained with the method of the invention represents another object of the present application.

A biological prosthesis, a medical device and particularly a cardiac prosthesis comprising the surface obtained according to the method of the present application represent further objects of the present invention.

A surface obtained with the pre-treatment and the treatment method of the invention represents another object of the present application.

A biological prosthesis, a medical device and a cardiac prosthesis comprising the surface obtained according to the pre-treatment and the treatment method of the present application represent further objects of the present invention

As per the present invention, the method for the treatment of a surface to be contacted with a biological fluid according to the invention is a method to preserve and maintain the proper structural biomechanical properties.

In particular, the method of the invention has proved to increase the elongation properties of the biological treated tissues.

In particular, said method preserves the collagen structure of the BHVs leaflets.

Accordingly, the method for the treatment of a surface to be contacted with a biological fluid according to the invention is a protective method for maintaining the proper physiological haemo- and fluid-dynamics properties of the treated BHVs.

A surface obtained with the method of the invention represents another object of the present application.

A biological prosthesis, a medical device and a cardiac prosthesis comprising the surface obtained according to the method of the present application represent further objects of the present invention.

A surface obtained with the pre-treatment and the treatment method of the invention represents another object of the present application.

A biological prosthesis, a medical device and a cardiac prosthesis comprising the surface obtained according to the pre-treatment and the treatment method of the present application represent further objects of the present invention.

As per the present invention, the method for the treatment of a surface to be contacted with a biological fluid according to the invention is an anti-microbial and an anti-viral method, in that it is a disinfecting method of the treated surface.

In particular, the anti-microbial method of the invention has a biocidal action.

More in particular, the antimicrobial method of the invention is active against microorganisms that are responsible for the onset of endocarditis.

In an embodiment, said microorganisms are Gram⁺ bacteria, Gram⁻ bacteria, yeasts and moulds.

In another embodiment, said microorganisms are mycobacteria, such as Mycobacterium chelonae.

Accordingly, the method for the treatment of a surface to be contacted with a biological fluid according to the invention is an anti Gram⁺ method, an anti-Gram⁻ method, an anti-yeast method, an antimould method, an anti-mycobacteria method.

In particular, the anti-viral method of the invention has a virucidal action against viruses.

More in particular, said virucidal action is according to the standard EN 14476.

In particular, said viruses belong to the families of Picornaviridae, Adenoviridae and Caliciviridae.

More in particular, said microorganisms are Poliovirus Type 1 LSc-2ab (RVB-1260), Adenovirus Type 5 (ATCC VR-5) and Murine norovirus strain S-99 (RVB-651).

A surface obtained with the method of the invention represents another object of the present application.

A biological prosthesis, a medical device and a cardiac prosthesis comprising the surface obtained according to the method of the present application represent further objects of the present invention.

A surface obtained with the pre-treatment and the treatment method of the invention represents another object of the present application.

A biological prosthesis, a medical device and a cardiac prosthesis comprising the surface obtained according to the pre-treatment and optionally the treatment method of the present application represent further objects of the present invention.

More, in particular, said biological prosthesis may be represented by a cardiovascular prosthesis obtained with the pre-treatment and optionally the treatment method of the invention.

According to a third object of the invention, it is disclosed a method for the treatment of a disease comprising the use of the medical device, the biological prosthesis or the cardiac prosthesis above disclosed.

As per a preferred aspect, said disease is a heart disease.

As per an aspect, said disease is in a human, while per another aspect, said disease is in an animal.

In a particular embodiment, the method for the treatment of the disease according to the invention comprises a valve-in-valve approach, wherein a valve is deployed inside a dysfunctional valve without its preliminary removal.

As a further object of the present invention, it is disclosed a solution comprising a phenolic compound or a mixture of phenolic compounds to be used in the method of the present application.

In particular, the preparation of said solution comprises a first step, wherein the phenolic compound is solubilized in an alcoholic solvent.

For instance, a phenolic compound from the above List A may be solved.

If necessary, a further phenolic compound can be solubilized in an isotonic buffered solution.

For instance, a phenolic compound from the above List B may be solved.

According to a preferred embodiment, the solution of compound A represents 10% (volume) of the final solution and the solution of compound B represents 90% (volume) of the final solution.

According to a preferred aspect, the preparation of the solution is carried out in the dark and preferably in the complete darkness, i.e. avoiding any exposure to light.

The present invention will be further described in connection with the following experimental section.

The following experimental session shows the results of assays carried out on surfaces treated according to the present invention.

### Polyphenolic Solutions

The following table reports some examples of polyphenolic solutions according to the invention.

| Solu- | | |
|---|---|---|
| tion | Component A | Component B |
| 1 | Resveratrol 3 ± 2 mg/ml | Tannic Acid 4 ± 3.5 mg/ml |
| 2 | Resveratrol 3 ± 2 mg/ml | Cynarin 2 ± 1.5 mg/ml |
| 3 | Aloin 1.5 ± 1 mg/ml | Epigallocatechin 2 ± 1 mg/ml |
| 4 | Aloin 1.5 ± 1 mg/ml | Chlorogenic Acid 4 ± 3 mg/ml |
| 5 | Caffeic Acid 2 ± 1.5 mg/ml | Tannic Acid 4 ± 3.5 mg/ml |
| 6 | Caffeic Acid 2 ± 1.5 mg/ml | Hydroxytyrosol 4 ± 2.5 mg/ml |
| 7 | Rosmarinic Acid 2.5 ± 2 mg/ml | Cynarin 2 ± 1 mg/ml |
| 8 | Naringenin 1 ± 0.5 mg/ml | Gallic Acid 1.5 ± 1 mg/ml |
| 9 | Hesperetin 2 ± 1.5 mg/ml | Gallic Acid 1.5 ± 1 mg/ml |

In particular, the surfaces have been treated with the Solution 5 according to the above disclosure.

### Preparation of the Polyphenolic Solution 5

Caffeic acid as Component A has been weighted according to the indicated concentrations and solubilized in ethanol to a 10% final volume of the polyphenol mixture. Tannic acid as the Component B column has been weighted according to the indicated concentrations and solubilized in modified phosphate buffer to 90% final volume of the polyphenol mixture. Both steps are performed in the dark. When solubilization is complete, the two solutions are admixed. pH is adjusted to 5-7. The solution is filtered with a 0.22 μm filter. This solution is referred to as Solution 5 or the Polyphenolic Solution.

Other solutions according to the present invention may be prepared in analogy with the above disclosure.

### Glutaraldehyde Stabilization

### Tissue Processing

Several bovine pericardia have been carefully selected to obtain rectangular patches (n=32). All patches were subjected to preliminary GLU crosslinking treatment. Briefly, pericardial tissue was incubated in a buffered GLU solution for three steps of 24 hs each in a dark room. For this example, a GLU solution is a 0.6%±0.5% v/v for the first and second steps followed by a 0.2%±0.15% v/v for the third ones. GLU treated pericardial patches were subjected to two washing steps in phosphate buffer for 15 minutes each.

Sixteen patches were incubated with Solution 5 of the invention prepared as above disclosed under moderate but constant stirring in the dark, for two-step of 25±10 minutes each, at room temperature (RT). At the end of the incubation, the treated patches were subjected to five washes in phosphate buffer for 15 to 30 minutes each. Samples are referred to as TREATED.

The remaining GLU fixed pericardial patches were adopted as control (GLU, n=16).

### Determination of the Content of Free Carboxyl Groups

The pericardial patches were included in OCT (Optimal Cutting Temperature) and frozen by immersion in isopentane pre-cooled in liquid nitrogen. Cryosections with a thickness of 7 μm were then produced using MirrIR slides suitable for infrared reflectance studies as support and analyzed by FT-IR microscopy in reflectance and mosaic mode with 64 scans for each selected area. The detector used is FPA with 4 cm⁻¹ resolution. The treated tissues showed a lower concentration of carboxyl groups at wave number 1233 cm⁻¹ (corresponding to the C—O bonds stretching of carboxyl groups) compared to samples fixed only in GLU. Considering the total of free carboxylic groups quantified in the GLU fixed samples as 100% of the available groups, treated samples reported a decrease equal to 76% of the total.

### Determination of the Content of Free Aldehydes Groups

Prepare 150 ml of Solution A: 0.2M Citric Acid, 0.5M Sodium Hydroxide and 8 mM Tin (II) Chloride in ultra-pure water. Prepare 25 ml of Solution B: 0.22M Ninhydrin dissolved in 25 ml of Monomethylether of Ethylene Glycol (Cellosolve) in a dark screw-cap bottle. Combine one volume of Solution A with an equal volume of Solution B and mix in the dark for 45 minutes to obtain Solution C. Please note that 2 ml of Solution C is required for each sample to be analyzed.

Tissue samples should be prepared to have a wet weight of approximately 20 mg each. Each sample is incubated at 100° C. in 2 ml of Solution C for 20 minutes in the dark, cooled in water and diluted with 15 ml of 50% Isopropanol. The developed colour is read at 570 nm within 30 minutes. The nmoles of aldehyde groups are determined with respect to a glycine standard.

Considering the total of free aldehyde quantified in the GLU fixed samples as 100% of the available groups, treated samples reported a decrease equal to 56.3% of the total.

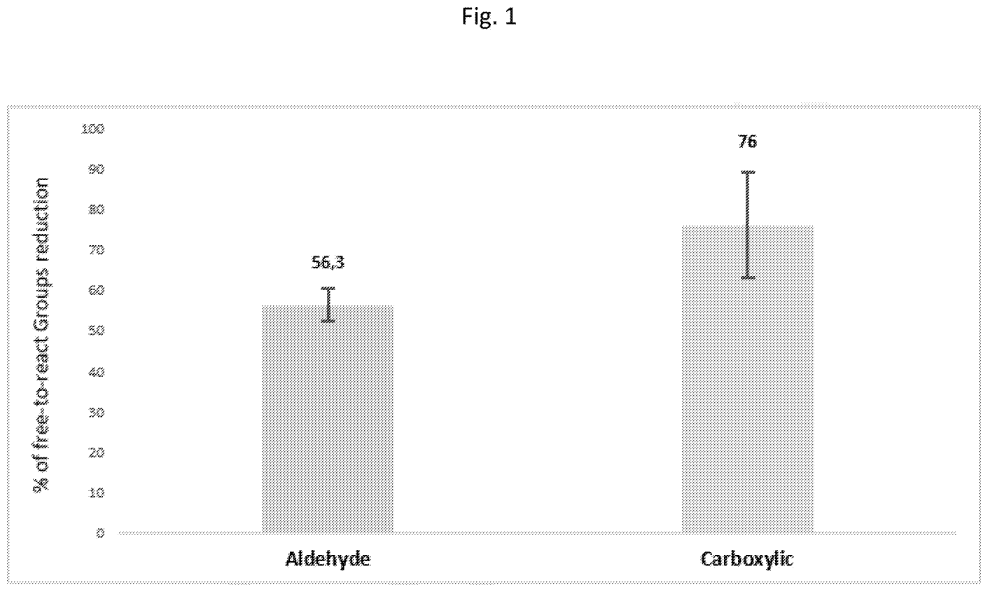

FIG. 1 shows the percentage of free-to-react aldehyde and carboxylic groups decrease in treated pericardial tissue patches (n=16 for each type of chemical group determination).

### Evaluation for Platelet Adhesiveness

### Tissue Processing

To assess the propensity for platelet adhesiveness and fibrin release under flow conditions, an in-vitro blood flow model was adopted. Several bovine pericardia have been carefully selected to obtain rectangular patches (n=12). All patches were subjected to preliminary GLU crosslinking treatment. Briefly, pericardial tissue was incubated in a buffered GLU solution for three steps of 24 hs each in a dark room. For this example, a GLU solution is a 0.6%±0.5% v/v for the first and second steps followed by a 0.2%±0.15% v/v for the third ones. GLU treated pericardial patches were subjected to two washing steps in phosphate buffer for 15 minutes each.

Six patches were incubated with the Solution 5 of the invention prepared as above disclosed under moderate but constant stirring in the dark, for two-step of 25±10 minutes each, at room temperature (RT). At the end of the incubation, the treated patches were subjected to five washes in phosphate buffer for 15 to 30 minutes each. Samples are referred to as TREATED.

The remaining GLU fixed pericardial patches were adopted as control (GLU, n=6).

### Platelets Adhesiveness Quantification

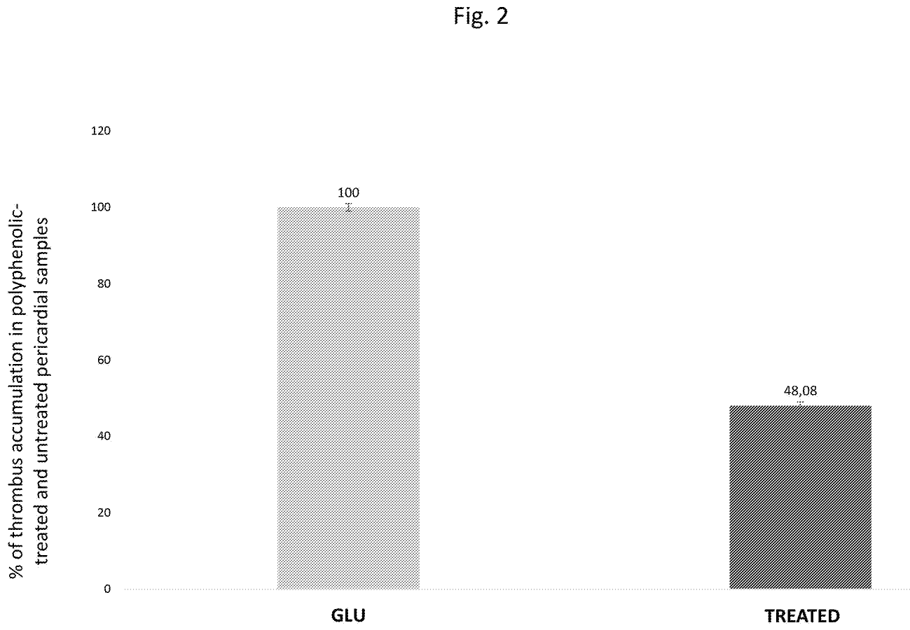

Heparinized bovine blood was collected from 3 different animals and radioisotope was added for thrombus quantification. Pericardial tissue strips were deployed in a 25.4 mm conduit and blood flow at 2.5 L/min was enabled with a peristaltic pump for 1 hour. The strips were rinsed with saline and placed in a gamma counter for quantification of radiation (reflective of relative thrombosis). The mean radiation values are 73.133 counts per minute (cpm) for Glu and 35.165 cpm for treated samples. In FIG. 2 results are expressed as a percentage of platelets propensity reduction in polyphenolic-treated tissues taking into account GLU samples as 100%.

### Mechanical Properties of the Treated Tissue

### Tissue Processing

Seventy-two rectangular strips (about 10 mm long and 8 mm wide) of bovine pericardia have been processed with the GLU solution as above disclosed (“Tissue processing”). GLU treated pericardial stripes were subjected to two washing steps in phosphate buffer for 15 minutes each. Thirty-six patches were incubated with Solution 5 above-disclosed under moderate but constant stirring in the dark, for two-step of 25±10 minutes each, at room temperature. At the end of the incubation, the treated patches were subjected to five washes in phosphate buffer for 15 to 30 minutes each. Samples are referred to as TREATED.

The remaining GLU fixed pericardial stripes were adopted as control (GLU, n=36).

### Mechanical Properties Evaluation

Each stripe was mounted on the tensile equipment to undergo a uniaxial tensile test fixed with rubber grips (Thumler Z3-X500 equipped with 100 N/500 N load cell). The sample initial length (distance between the grips) was set as 50 mm. The tensile test was run at 50 mm/min.

Each stripe was dimensionally characterized in terms of length (useful length 50 mm), width and thickness (average value of the measurements) and the cross-sectional area was calculated (with x thickness). The following parameters were obtained from each tensile curve:
Breaking strength [N], in terms of maximum strength before failure;
Ultimate tensile strength [MPa], in terms of maximum strength divided by the cross-sectional area (UTS);
Failure strain [%], in terms of strain at the maximum strength;
Young's Modulus [MPa], in terms of the slope of the linear region of the stress-strain curve (elastic phase);
   For each parameter, the average value was calculated from the samples, obtaining one value for each patch. The average value was calculated again from the patches, obtaining one value for each test group.

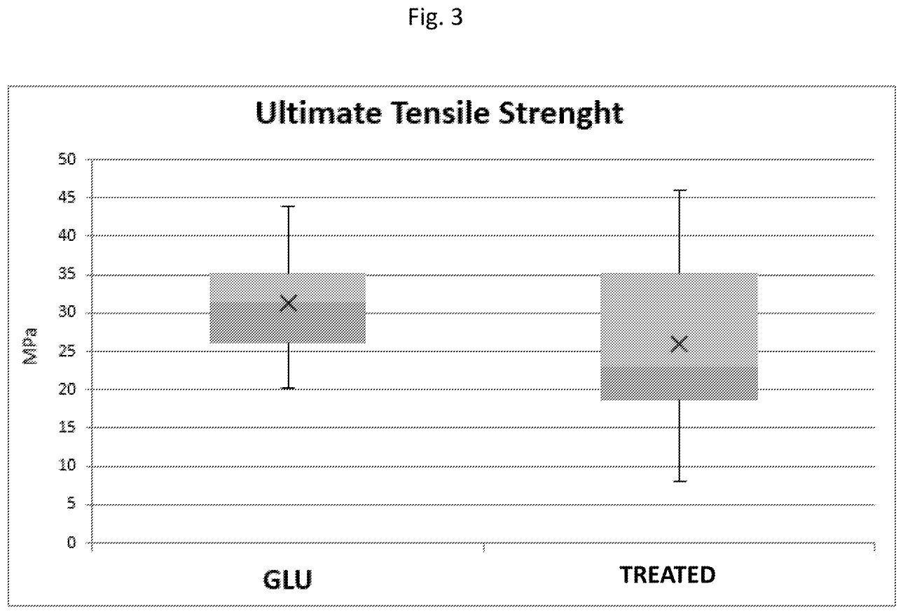

FIG. 3 shows the ultimate Tensile Strength comparison between treated and untreated samples (GLU). The Ultimate Tensile Strength (UTS), often shortened to tensile strength (TS), is the maximum stress that a material can withstand while being stretched or pulled before breaking. The polyphenolic treatment does not report a statistically significant difference from the control sample (GLU).

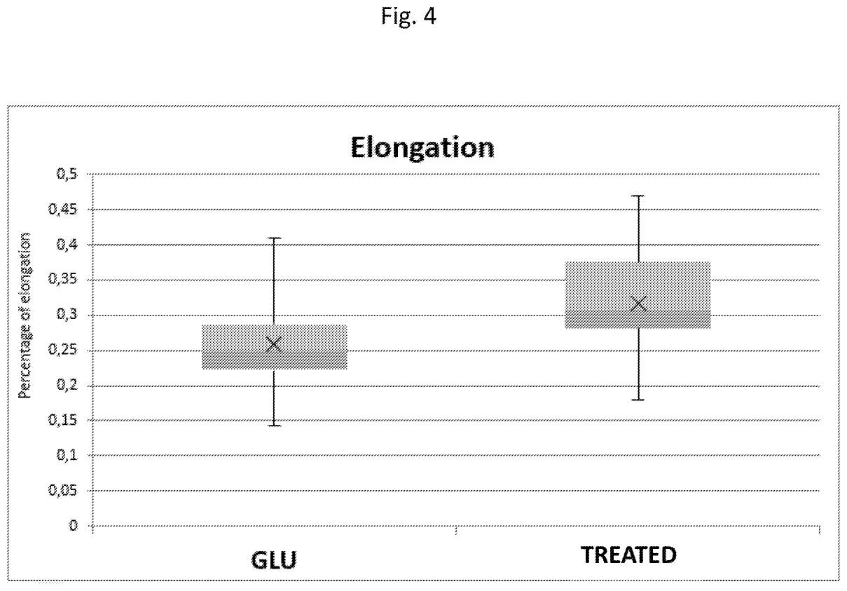

FIG. 4 shows the percentage of elongation comparison between treated and untreated samples (GLU).

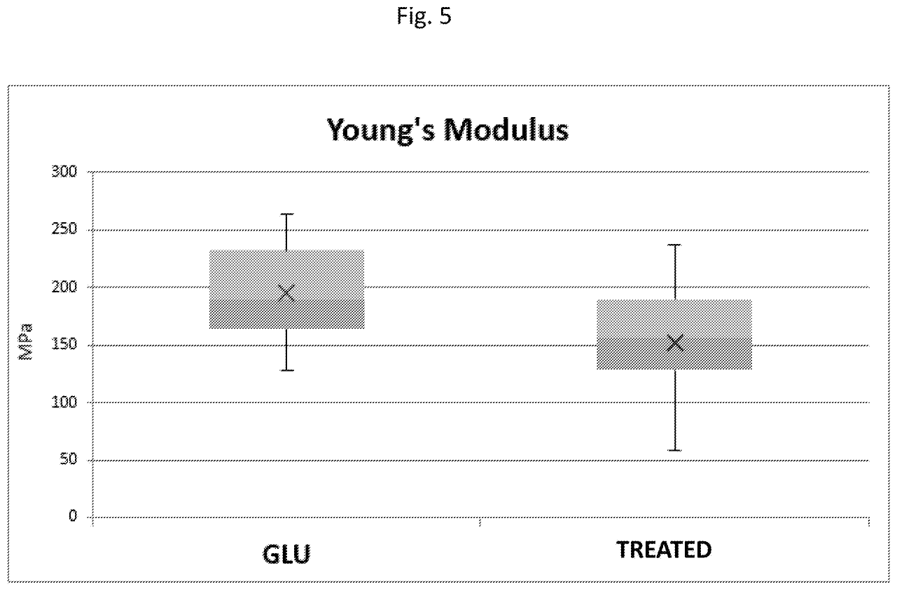

FIG. 5 shows Young's Modulus correlation between treated and untreated samples (GLU). The percentage of elongation is strictly related to Young's Modulus. The Young's Modulus or Modulus of Elasticity is a feature, characteristic of a material, which expresses the relationship between tension and deformation in the case of uniaxial load conditions and the case of elastic (reversible) behaviour of the material. It is defined as the ratio between the applied stress and the resulting deformation. As the Young's Modulus increases, also the stiffness of the material increases. The increase in the elasticity level of the treated tissue leads to a consequent decrease in its Young's Modulus compared to the untreated pericardium (GLU).

The gain in elasticity allows a better distribution of the mechanical load, especially to the advantage of the BHVs areas subjected to greater pressure. This avoids the formation of tears and preserves the collagen structure of the valve leaflets.

### The Bactericidal Potential of the Polyphenolic Solution

The Bactericidal Activity (BA) of the polyphenolic solutions of the invention was evaluated regarding the following different micro-organism: Staphylococcus aureus ATCC 6538, Pseudomonas aeruginosa ATCC 9027, Enterococcus faecalis ATCC 29212, Listeria monocytogenes ATCC 19111, Salmonella enterica typhimurium ATCC 14028, Streptococcus viridans ATCC 6249, a nontuberculous mycobacterium Mycobacterium chelonge ATCC 35752, a yeast Candida albicans ATCC 10231 and a fungus Aspergillus brasiliensis ATCC 16404.

BA assay consists of a suspension method with a single incubation for 24 hours of the bacteria with a known concentration of the Solution 5 of the invention (inoculum). At the end of the incubation time, the content of each test tube is seeded into 90 mm sterile Petri dishes in a specific agar medium, with pour plate or spread plate technique depending on the micro-organisms tested, after dilution in Tryptone Salt Broth (MRD Broth). Then the plates obtained are incubated at specific conditions and temperatures according to the growth requirements of each micro-organism.

### Preparation of the Inoculum

For each type of micro-organism, a microbial suspension in MRD Broth was quantified through the spectrophotometer at 620 nm wavelength in disposable 10 mm path length cuvette. The absorbance of an aliquot part of the suspension is measured: the range between 0.150 and 0.460 corresponds to a concentration of cells between 1×10\hat{ }8 CFU/ml and 3×10\hat{ }8 CFU/ml (with Candida albicans between 1×10\hat{ }7 CFU/ml and 3×10\hat{ }7 CFU/ml). For Streptococcus oralis, as there was no correlation between the absorbance measure and the bacteria concentration, the quantification was performed by cell count at the microscope.

Data were elaborated comparing the growth on the sample to the bacteria concentration at t0, to see an effect in growth promotion or inhibition, and expressed as a percentage of bactericidal activity. Results were compared to control samples (antibiotics and ethanol solutions).

The table here below reports the percentage of bactericidal activity compared to standard antibodies and ethanol solution.

| | | | BACTERICIDAL ACTIVITY % | | |
|---|---|---|---|---|---|
| | ATCC | | SOLUTION | | |
| MICRO-ORGANISM | Code | FAMILY | 5 | ANTIBIOTIC | ETHANOL |
| Pseudomonas aeruginosa | 9027 | GRAM⁻ | 99.999 | 99.999 | 99.99 |
| Salmonella enterica | 14028 | GRAM⁻ | 99.999 | 99.999 | 90 |
| typhimurium | | | | | |
| Enterococcus faecalis | 29212 | GRAM⁺ | 99.99 | 99.999 | 0 |
| Listeria monocytogenes | 19111 | GRAM⁺ | 99.999 | 99.999 | 0 |
| Staphylococcus aureus | 6538 | GRAM⁺ | 99.999 | 99.999 | 99 |
| Streptococcus viridans | 6249 | GRAM⁺ | 99.999 | 99.999 | 90 |
| Candida albicans | 10231 | Yeast | 99.999 | 99.999 | 99.9 |
| Aspergillus brasiliensis | 16404 | Fungi | 99.9 | 99.999 | 90 |
| Mycobacterium chelonae | 35752 | Mycobacterium | 99.999 | 99.999 | 99 |

### The Virucidal Potential of the Polyphenolic Solution

The virucidal activity of the polyphenolic Solution 5 was assessed according to the guidelines: Test method and requirements European Standard EN 14476: 2013+A2: 2019/UNI EN 14476:2019—Chemical disinfectants and antiseptics. Quantitative suspension test for the evaluation of virucidal activity in the medical area. Test method and requirements (Phase 2/Step 1). The virucidal assay was performed regarding the following viruses strain: Poliovirus Type 1 LSc-2ab (RVB-1260), Adenovirus Type 5 (ATCC VR-5) and Murine norovirus S99 (RVB-651). The table here below reports the percentage of virucidal activity of the polyphenolic Solution 5 diluted to 80% (corresponding to the highest possible concentration assessable according to the method).

| | Percentage of virus inactivation |
|---|---|
| Poliovirus Type 1 LSc-2ab - RVB1260 | 99.00 ± 3.20 |
| Adenovirus Type 5 - ATCC VR5 | 99.70 ± 1.02 |
| Murine norovirus S99 - RVB651 | 99.48 ± 1.54 |

### Anti-Adhesiveness Effect of the Polyphenolic Solution on Different Strains of Micro-Organisms

The anti-adhesive activity on the treated surfaces was assessed with reference to the following different micro-organism: Staphylococcus aureus ATCC 6538, Pseudomonas aeruginosa ATCC 9027, Enterococcus faecalis ATCC 29212, Listeria monocytogenes ATCC 19111, Salmonella enterica typhimurium ATCC 14028, Streptococcus viridans ATCC 6249, a nontuberculous mycobacterium Mycobacterium chelonge ATCC 35752, a yeast Candida albicans ATCC 10231 and a fungus Aspergillus brasiliensis ATCC 16404.

Each strain of micro-organism was grown overnight in dedicated broth at 37° C. At the end of the incubation, the units forming colonies (UFC) were counted to determine the effective concentration of the microorganism.

### Tissue Processing

90 samples of approximately 2 cm² each of bovine pericardia have been processed with the GLU solution as above disclosed (“Tissue processing”). GLU treated pericardial samples were subjected to two washing steps in phosphate buffer for 15 minutes each. At the end of the incubation, 45 patches have been treated with the Solution 5 of the invention according to the disclosed method and then were subjected to five washes in phosphate buffer for 15 to 30 minutes each. These samples are referred to as TREATED.

The remaining GLU fixed pericardial specimens were adopted as control (GLU).

### Anti-Adhesiveness Effect Assessment

Polyphenolic-treated and untreated patches were washed with PBS and incubated overnight at room temperature in PBS+antibiotics (300 μg/mL). Different types of antibiotics specific to each type of microorganism strain were used (neomycin, penicillin, cephalosporin, polymyxin, rifamycin, lipiarmycin, quinolone, sulfonamide, macrolide, lincosamide, tetracycline, aminoglycoside, doxycycline, minocycline, ampicillin, amoxicillin/clavulanic acid, azithromycin, carbapenems, piperacillin/tazobactam, quinolones, chloramphenicol, ticarcillin, trimethoprim/sulfamethoxazole). After overnight incubation, the tissue patches were washed extensively in PBS to remove any trace of unbound antibiotic. Subsequently, the treated and untreated samples were exposed singularly to the different strains of micro-organism strain (micro-organism load 1×10⁷ CFU/mL) for 90 minutes at room temperature under moderate but constant agitation. At the end of the incubation, the tissue samples were subjected to three moderate vortexing passages to facilitate the detachment of the loosely bound bacteria. Finally, the samples were homogenized by Stomacher® 400 and serial dilutions of the obtained homogenates, plated in Petri dishes containing the appropriate selective growth media. After 24 hours of incubation at 37° C., the colony-formed units were counted for each type of sample.

Results were expressed as the percentage decrease of the attached micro-organisms assessed in the treated pericardial patches by comparison with untreated GLU fixed pericardial patches (n=5 for each micro-organism strain).

The table here below reports the percentage decrease of the attached micro-organisms assessed in treated pericardial patches. The percentage value was determined by a comparison between treated and untreated GLU fixed pericardial patches (n=5 for each micro-organism strain).

| | | | % of micro- |
|---|---|---|---|
| MICRO- | ATCC | | organism |
| ORGANISM | Code | FAMILY | removal |
| Pseudomonas aeruginosa | 9027 | GRAM⁻ | 90.4 ± 1.2 |
| Salmonella enterica | 14028 | GRAM⁻ | 93.2 ± 2.3 |
| typhimurium | | | |
| Enterococcus faecalis | 29212 | GRAM⁺ | 92.8 ± 4.6 |
| Listeria monocytogenes | 19111 | GRAM⁺ | 94.4 ± 4.8 |
| Staphylococcus aureus | 6538 | GRAM⁺ | 95.3 ± 3.8 |
| Streptococcus viridans | 6249 | GRAM⁺ | 96.1 ± 7.2 |
| Candida albicans | 10231 | Yeast | 91.7 ± 3.2 |
| Aspergillus brasiliensis | 16404 | Fungi | 93.6 ± 4.8 |
| Mycobacterium chelonae | 35752 | Mycobacterium | 92.9 ± 1.1 |

### Non-Pyrogenicity of the Polyphenolic-Treated Tissue

The monocyte activation test (MAT) has been qualified and validated for the detection of pyrogens by the European Center for the Validation of Alternative Methods (ECVAM) in 2005 and by the Interagency Coordinating Committee on the Validation of Alternative Methods (ICCVAM) in 2008. It has been among the compendial methods for pyrogen detection in the European Pharmacopeia since 2010 (Chapter 2.6.30) and mentioned by the FDA “Guidance For Industry—Pyrogen and Endotoxins testing: Questions and Answers”. The monocyte activation test (MAT) is the human in-vitro alternative to the rabbit pyrogen test (RPT) and allows the detection of the full range of pyrogens, including endotoxins and non-endotoxin pyrogens (NEPs).

### Tissue Processing

Eight-teen samples of bovine pericardia of approximately 2 cm² each have been processed with the GLU solution as above disclosed (“Tissue processing”). GLU treated pericardial samples were subjected to two washing steps in phosphate buffer for 15 minutes each. Nine patches were incubated with the Solution 5 under moderate but constant stirring in the dark, for two-step of 25±10 minutes each, at room temperature. At the end of the incubation, the treated patches were subjected to five washes in phosphate buffer for 15 to 30 minutes each. Samples are referred to as TREATED.

The remaining nine GLU fixed pericardial specimens were adopted as control (GLU, n=9).

### Pyrogenicity Assessment

Samples were placed for 1 hour in 40 ml of endotoxin-free water under moderate shaking at 37° C. The water was analyzed with the MAT test. Briefly, water was brought into contact with human monocytic cells, it will mimic what happens in the human body: in presence of pyrogens, the monocytes are activated and produce several types of cytokines including interleukin-6 (IL6). The cytokines are then detected using an immunological assay (ELISA) involving specific antibodies and an enzymatic colour reaction.

The table below reports the Endotoxin Unit evaluation in treated and untreated (GLU) pericardial patches. Reference Value to be considered pyrogenic: NMT 20 EU/device.

| Tissue type | Endotoxin unit/sample |
|---|---|
| GLU | <0.666 |
| TREATED | <0.666 |

Given the above-reported disclosure, there will be evident the advantages of the method of the present invention.

In particular, the method of the invention has proved not to alter the other properties of the surfaces treated and optionally pre-treated according to the above disclosure, and of the medical devices, biological prosthesis and particularly cardiac prosthesis comprising said surfaces.

As another advantage, the disclosed method has shown to inactivate the available aldehydic and carboxylic reacting groups on the treated or pre-treated surface.

In addition, as the platelets deposition on the fibrin network is responsible for the onset of subclinical leaflets thrombosis (SLT), which in turn is responsible for altering the mobility of the valve leaflets, the method of the invention prevent the impairment of the functionality of the BHVs.

It has been found that the protective action of the method of the invention is responsible for a better distribution of the mechanical load, which avoids the formation of tears, abrasion and holes.

As a further advantage, the method of the invention prevents the adhesion of microorganisms and the formation of biofilms on the treated and optionally pre-treated surfaces.

Again, the disclosed method avoid the bacterial and viral contamination of a surface treated and optionally pre-treated according to the present invention.

As a still further advantage, the disclosed method maintains the non-pyrogenic characteristic of the treated and optionally pre-treated surfaces.

What is claimed is:

1. A method for the treatment of a surface to be contacted with biological fluids, said method comprising contacting said surface with a solution comprising a phenolic compound or a mixture of phenolic compounds.

2. The method of claim 1, wherein said surface is a surface of a medical device.

3. The method of claim 1, wherein said surface is a surface of a biological prosthesis.

4. The method of claim 1, wherein said surface is a surface of one of a cardiac prosthesis, a cardiac valve, a pericardial tissue patch, or a surgical heart valve.

5. The method of claim 1, wherein said biological fluids are selected from the group comprising consisting of: blood, serum, plasma, vitreous gel, tears, urine, saliva, faeces, and synovial, peritoneal, pericardial, pleural and amniotic fluid.

6. The method of claim 1, wherein said phenolic compound or said mixture of phenolic compounds are selected from the group consisting of: phenols, phenolic aldehydes, phenolic acids, phenylamines, phenol compounds, flavonoids, phenylpropanoids and tannins.

7. The method of claim 1, wherein said phenolic compound or said mixture of phenolic compounds are selected from the group consisting of: vanillin, cinnamic acids, phenylalanine, coumarins, xanthones, catechins, flavonons, flavones, chalcones, flavanonols, flavanols, leucoanthocyanidin, anthocyanidin, and hydroxycinnamic acids.

8. The method of claim 1, wherein said phenolic compound or said mixture of phenolic compounds is selected from the group consisting of: resveratrol, aloin, cyanarin, epigallocatechin, tannic acid, caffeic acid, chlorogenic acid, hydroxytyrosol, rosmarinic acid, narigenin, gallic acid, hesperitin, quinic acid, eleonolic acid, pinoresinol, luteolin, apigenin, tangeritin, isorhamnetin, kaempferol, myricetin, eriodictyol, hesperetin, naringenin, theaflavin, thearubigins, daidzein, genistein, glycitein, pterostilbene, delphinidin, malvidin, pelargonidin, peonidin, chicoric acid, ferulic acid, and salicylic acid.

9. The method of claim 1, wherein said method comprises contacting said surface with the solution comprising the phenolic compound or the mixture of phenolic compounds for a period of time of less than 2 hours.

10. The method of claim 1, wherein said method comprises a first contacting step and a second contacting step, and wherein between said first and second contacting steps a rinsing step is performed.

11. The method of claim 1, wherein said method comprises a pre-treatment step with one or more compounds selected from the group consisting of glutaraldehyde, formaldehyde, quercetin, and genipin.

12. The method of claim 11, wherein said pre-treatment step comprises removing phospholipids.

13. The method of claim 12, wherein said pre-treatment step further comprises a preparing step comprising use of a capping agent.

14. The method of claim 1, wherein said method is one or more of: a method to preserve and maintain proper structural biomechanical properties, a protective method for maintaining proper physiological haemo- and fluid-dynamics properties of a treated surface, an anti-microbial and an anti-viral method for disinfecting a treated surface.

15. A surface to be contacted with biological fluids obtained according to the method of claim 1.

16. A medical device or a biological prosthesis comprising the surface of claim 15.

17. The medical device of claim 16, wherein said medical device is selected from the group consisting of: cardiac valve, tendon, ligament, pericardium, muscular fasciae, dura mater, tympanic membrane, intestinal submucosa, cartilage, adipose and bone tissue, pelvic, abdominal, breast and dermal tissue.

18. A method for the treatment of a disease, the method comprising using the medical device or the biological prosthesis of claim 16.

19. The method of claim 18, wherein said disease is a heart disease.

20. The method of claim 18, wherein said disease is in humans or in animals.

21. The method of claim 18, wherein said method comprises a valve-in-valve approach.

22. A biocidal method for the treatment of a surface to be contacted with biological fluids, said method comprising contacting said surface with a solution comprising a phenolic compound or a mixture of phenolic compounds.

23. The biocidal method of claim 22, wherein said surface is a surface of a medical device.

24. The biocidal method of claim 22, wherein said surface is a surface of a biological prosthesis.

25. The biocidal method of claim 22, wherein said surface is a surface of one of a cardiac prosthesis, a cardiac valve, a pericardial tissue patch, or a surgical heart valve.

26. The biocidal method of claim 22, wherein said biological fluids are selected from the group consisting of: blood, serum, plasma, vitreous gel, tears, urine, saliva, faeces, and synovial, peritoneal, pericardial, pleural and amniotic fluid.

27. The biocidal method of claim 22, wherein said phenolic compound or said mixture of phenolic compounds are selected from the group consisting of: phenols, phenolic aldehydes, phenolic acids, phenylamines, phenol compounds, flavonoids, phenylpropanoids and tannins.

28. The biocidal method of claim 22, wherein said phenolic compound or said mixture of phenolic compounds are selected from the group consisting of: vanillin, cinnamic acids, phenylalanine, coumarins, xanthones, catechins, flavonons, flavones, chalcones, flavanonols, flavanols, leucoanthocyanidin, anthocyanidin, and hydroxycinnamic acids.

29. The biocidal method of claim 22, wherein said phenolic compound or said mixture of phenolic compounds is selected from the group consisting of: resveratrol, aloin, cyanarin, epigallocatechin, tannic acid, caffeic acid, chlorogenic acid, hydroxytyrosol, rosmarinic acid, narigenin, gallic acid, hesperitin, quinic acid, eleonolic acid, pinoresinol, luteolin, apigenin, tangeritin, isorhamnetin, kaempferol, myricetin, eriodictyol, hesperetin, naringenin, theaflavin, thearubigins, daidzein, genistein, glycitein, pterostilbene, delphinidin, malvidin, pelargonidin, peonidin, chicoric acid, ferulic acid, and salicylic acid.

30. The biocidal method of claim 22, wherein said method comprises contacting said surface with the solution comprising the phenolic compound or the mixture of phenolic compounds for a period of time of less than 2 hours.

31. The biocidal method of claim 22, wherein said method comprises a first contacting step and a second contacting step, and wherein between said first and second contacting steps a rinsing step is performed.

32. The biocidal method of claim 22, wherein said method comprises a pre-treatment step with one or more compounds selected from the group consisting of; glutaraldehyde, formaldehyde, quercetin, and genipin.

33. The biocidal method of claim 32, wherein said pre-treatment step comprises removing phospholipids.

34. The biocidal method of claim 33, wherein said pre-treatment step further comprises a preparing step comprising use of a capping agent.

35. The biocidal method of claim 22, wherein biocidal activity is against microorganisms that are responsible for the onset of endocarditis.

36. The biocidal method of claim 35, wherein said microorganisms are Gram⁺ bacteria, Gram⁻ bacteria, yeasts, moulds or viruses.

37. The biocidal method of claim 35, wherein said microorganisms are mycobacteria.

38. The biocidal method of claim 36, wherein said viruses belong to the families Picornaviridae, Adenoviridae and Caliciviridae.

39. A surface obtained with the biocidal method of claims 22.

40. A biological prosthesis, a medical device and a cardiac prosthesis comprising the surface of claim 39.

41. The biological prosthesis of claim 40, which is represented by a cardiovascular prosthesis.
